(19) United States
(12) Patent Application Publication
(10) Pub. No.: US 2023/0030607 A1
(43) Pub. Date: Feb. 2, 2023

(54) Novel oxygen pulse therapy method for treating COVID19 and viral, bacterial, fungal or parasitic respiratory and other diseases

(71) Applicant: Jose Octavio Hernandez, Santiago (CL)

(72) Inventor: Jose Octavio Hernandez, Santiago (CL)

(21) Appl. No.: 17/382,410

(22) Filed: Jul. 22, 2021

(51) Int. Cl. A61M 16/00; A61M 16/10

(52) U.S. Cl. CPC A61M 16/0012; A61M 16/1005

(57) ABSTRACT

A novel oxygen pulse therapy methods of treating subjects suffering from COVID19 and other respiratory and others diseases are provided. Aspects of the methods including administering to the subjects an effective amount of oxygen as pulses through the respiratory tract are included. Also provided are methods of assessing severity of the disease, mild, moderate, severe, or critical, and oxygen doses and frequencies. The method can be applied to viral, bacterial, fungal or parasitic respiratory and others diseases infections such as corona virus (SARS-CoV-2), influenza virus such as influenza A or rotavirus, bacterial pneumonia and meningitis such as Streptococcus pneumonia or parasitic and other diseases such AIDS, Ebola, tuberculosis and malaria.

### FIELD OF THE INVENTION

This invention relates to medical treatments and composition and procedures useful therein. More specifically, it relates to a therapy of oxygen pulses delivered to the pulmonary system of human patient for treating viral, bacterial, fungal or parasitic or other diseases such as corona virus (SARS-CoV-2), influenza virus such as Influenza virus A, Influenza virus B, Influenza virus C or rotavirus, bacterial pneumonia and meningitis such as Streptococcus pneumonia or parasitic and other diseases such AIDS, Ebola, tuberculosis and malaria.

### BACKGROUND OF THE INVENTION

COVID-19 is the disease caused by a new coronavirus called SARS-CoV-2. WHO first learned of this new virus on 31 Dec. 2019, following a report of a cluster of cases of ‘viral pneumonia’ in Wuhan, People's Republic of China.

SARS-CoV-2 is a positive-sense single-stranded RNA virus (and hence Baltimore class IV) that is contagious in humans. As described by the US National Institutes of Health, it is the successor to SARS-CoV-1, the strain that caused the 2002-2004 SARS outbreak.

Taxonomically, SARS-CoV-2 is a strain of severe acute respiratory syndrome-related coronavirus (SARSr-CoV). It is believed to have zoonotic origins and has close genetic similarity to bat coronaviruses, suggesting it emerged from a bat-borne virus. There is no evidence yet to link an intermediate host to its introduction to humans. The virus shows little genetic diversity, indicating that the spillover event introducing SARS-CoV-2 to humans is likely to have occurred in late 2019.

The most common symptoms of COVID-19 are Fever, Dry cough, Fatigue

Other symptoms that are less common and may affect some patients include:
Loss of taste or smell,
Nasal congestion,
Conjunctivitis (also known as red eyes)
Sore throat,
Headache,
Muscle or joint pain,
Different types of skin rash,
Nausea or vomiting,
Diarrhea,
Chills or dizziness.

Symptoms of severe COVID-19 disease include:
Shortness of breath,
Loss of appetite,
Confusion,
Persistent pain or pressure in the chest,
High temperature (above 38° C.).

Other less common symptoms are:
Irritability,
Confusion,
Reduced consciousness (sometimes associated with seizures),
Anxiety,
Depression,
Sleep disorders,
More severe and rare neurological complications such as strokes, brain inflammation, delirium and nerve damage.

People of all ages who experience fever and/or cough associated with difficulty breathing or shortness of breath, chest pain or pressure, or loss of speech or movement should seek medical care immediately. If possible, call your health care provider, hotline or health facility first, so you can be directed to the right clinic.

Scientists around the world are working to find and develop treatments for COVID-19.

Optimal supportive care includes oxygen for severely ill patients and those who are at risk for severe disease and more advanced respiratory support such as ventilation for patients who are critically ill.

Dexamethasone is a corticosteroid that can help reduce the length of time on a ventilator and save lives of patients with severe and critical illness

Results from the WHO's Solidarity Trial indicated that remdesivir, hydroxychloroquine, lopinavir/ritonavir and interferon regimens appear to have little or no effect on 28-day mortality or the in-hospital course of COVID-19 among hospitalized patients.

Hydroxychloroquine has not been shown to offer any benefit for treatment of COVID-19

U.S. Pat. No. 11,013,687 presents a novel preventive treatment for COVID 19 and therapeutic treatment for early stages of COVID 19 or any other disease caused by SARS CoV 2. Pharmaceutical formulations and devices are disclosed to be able to implement these treatments. These treatments to prevent and alleviate the symptoms at early stages of COVID 19, the pandemic disease caused by SARS CoV 2. These treatments are based on the administration the nasal cavity and/or the lungs of solutions containing iota Carrageenan with or without the addition of xylitol as antiviral drug substances.}

U.S. Pat. No. 10,980,756 presents disclosure features compounds and compositions that are useful in methods of treating coronavirus infections (e.g., useful in methods of treating COVID-19), methods include administering to the subject niclosamide compounds (or pharmaceutically acceptable salts and/or co-crystals thereof, e.g., niclosamide).

U.S. Pat. No. 10,975,139 presents disclosure that provides antibodies and antigen-binding fragments thereof that bind specifically to a coronavirus spike protein and methods of using such antibodies and fragments for treating or preventing viral infections (e.g., coronavirus infections).

U.S. Pat. No. 10,973,910 presents neoglycoconjugates as immunogens and therapeutic/diagnostic tools are described herein. Applications of the neoglycoconjugates as antigens, immunogens, vaccines, and in diagnostics are also described. Specifically, the use of (neo)glycoconjugates as vaccine candidates and other therapeutic tools against cancers, viruses such as SARS-CoV-2, and other diseases characterized by expression of aberrant glycosylation.

U.S. Pat. No. 10,973,908 presents a live genetically engineered bacterium, comprising a genetically engineered construct comprising a nucleic acid sequence encoding at least one portion of a SARS-CoV-2 antigen, the live genetically engineered bacterium being adapted for administration to a human or animal and colonization of at least one tissue under non-lethal conditions. The antigen is preferably the SARS-CoV-2 spike protein. The nucleic acid sequence preferably includes an associated promoter.

U.S. Pat. No. 10,967,182 presents systems and methods for reducing pulmonary inflammation and/or increasing bronchial compliance in a patient utilize transcutaneous stimulation of neural structures in a region of an ear of a patient delivered by an auricular stimulation device having an in-ear component with a first electrode disposed in a patient's ear and an earpiece component with a second electrode placed around the auricle. A pulse generator may control delivery of therapy by delivering both a first series of stimulation pulses to the first electrode for stimulating a first neural structure(s) and a second series of stimulation pulses to the second electrode for stimulating second neural structure(s). The first and second electrodes are in non-piercing contact with tissue on and/or surrounding the ear. The systems and methods may be used to treat viral or bacteria infections, such as SARS, MERS, or COVID-19.

U.S. Pat. No. 10,959,969 provides methods for killing the SARS CoV-2 virus in mammals and treating the Coronavirus Disease-19 (Covid-19) in mammals including humans using compositions including protocatechuic acid. The present disclosure provides methods and pharmaceutical and nutraceutical compositions that reduce or substantially eliminate the SARS CoV-2 virus in mammals. In one embodiment, a method of treating a mammal with Covid-19 is provided comprising administering protocatechuic acid to a mammal in need of such treatment a therapeutically effective amount of a protocatechuic acid composition.

U.S. Pat. No. 10,954,289 provides antibodies and antigen-binding fragments thereof that bind specifically to a coronavirus spike protein and methods of using such antibodies and fragments for treating or preventing viral infections (e.g., coronavirus infections)

U.S. Pat. No. 10,925,889 presents a method of treating, reducing, or alleviating a medical condition in a patient is disclosed herein. The method includes administering to a patient in need thereof a biocompatible drug comprising one or more antiviral medications together with one or more cell pathway inhibitors dissolved in a non-toxic semifluorinated alkane, the patient having one or more respiratory tract inflammatory diseases, the one or more cell pathway inhibitors blocking an inflammatory response of inflamed tissue without inhibiting an immune response of the patient, and the semifluorinated alkane evaporating quickly upon administration to the patient so as to leave the biocompatible drug at a desired treatment location

U.S. Pat. No. 10,925,806 presents a device for acupuncture and moxibustion therapy and uses. The device utilizes energy waves for preventing, alleviating or treating COVID-19 (SARS-CoV-2) coronavirus infection.

U.S. Pat. No. 10,918,633 presents a method of treating coronavirus by administering a pharmaceutical composition containing a therapeutically effective amount of isomyosmine or a pharmaceutically acceptable salt thereof.

U.S. Pat. No. 10,875,855 present methods of inhibiting the replication of influenza viruses in a biological sample or patient, of reducing the amount of influenza viruses in a biological sample or patient, and of treating influenza in a patient, comprises administering to said biological sample or patient an effective amount of A compound of Formula (VII): ##STR00123## or a pharmaceutically acceptable salt thereof, wherein X.sup.1 is —F, —Cl, —CF.sub.3, —CN, or —CH.sub.3; X.sup.2 is —H, —F, or —Cl; Z.sup.1 is N or CH; Z.sup.2 is N or CR.sup.0; R.sup.0 is —H, —F, or —CN; R is —H or C.sub.1-4alkyl; R.sup.1 is —CH.sub 0.3, —CH.sub.2F, —CF.sub.3, —C.sub.2H.sub.5, —CH.sub.2CH.sub.2F, or —CH.sub.2CF.sub.3; R.sup.4 and R.sup.5 are each —H; and ring P is 3-6 membered carbocyclic ring.

U.S. Pat. No. 10,874,687 is the use of a small group of purine nucleotide phosphoramidate disclosed herein or a pharmaceutically acceptable salt thereof in an effective amount for the treatment or prevention of the novel 2019 coronavirus disease (COVID-19) in a host, for example a human, in need thereof.

U.S. Pat. No. 10,874,684 present compositions and method disclosed for treating ARDS. In particular, disclosed a composition that contains one, two, or more cytidine diphosphate (CDP)-conjugated precursors selected from the group consisting of CDP-choline, CDP-ethanolamine, and CDP-diacylglycerol (CDP-DAG) in a pharmaceutically acceptable carrier for use in treating ARDS.

U.S. Pat. No. 10,828,336 present a cell based therapy that comprises administration to the lung by injection into the blood system of viable, mammalian cells effective for alleviating or inhibiting pulmonary disorders. The cells may express a therapeutic transgene or the cells may be therapeutic in their own right by inducing regenerative effects.

U.S. Pat. No. 10,729,735 presents a method of treating viral infection, such as viral infection caused by a virus of the Coronaviridae family, is provided. A composition having at least oleandrin is used to treat viral infection.

United States Patent Application 20200390888 present a non-replicating recombinant adeno-associated virus (rAAV) having an AAV capsid having packaged therein a vector genome which comprises AAV inverted terminal repeat sequences and at least one nucleic acid sequence encoding four different immunoglobulin regions (a), (b), (c) and (d) is provided. The rAAV-expressed immunoglobulins are useful for providing passive immunization against influenza A and influenza B. Also described herein are compositions containing the rAAV. Methods of vaccinating patients against influenza are provided.

United States Patent Application 20200362044 present methods of treating a subject suffering from COVID-19 are provided. Aspects of the methods including administering to the subject an effective amount of an inhibitor of CCR5/CCL5 interaction, such as a CCR5 antagonist. Also provided are methods of assessing severity of a disease involving hypercytokinemia, such as COVID-19, by determining the level of CCL5/RANTES in a subject, as well as compositions for use in such methods.

AIDS is an infection that attacks the body's immune system specifically the white blood cells called CD4 cells. HIV destroys these CD4 cells weakening a person's immunity against infections such as tuberculosis and some cancers

The most advanced stage of HIV infection is acquired immunodeficiency syndrome (AIDS), which can take many years to develop if not treated, depending on the individual. AIDS is defined by the development of certain cancers, infections or other severe long-term clinical manifestations.

The human immunodeficiency virus (HIV) targets the immune system and weakens people's defense against many infections and some types of cancer that people with healthy immune systems can fight off. As the virus destroys and impairs the function of immune cells, infected individuals gradually become immunodeficient. Immune function is typically measured by CD4 cell count.

AIDS continues to be a major global public health issue, having claimed 34.7 million [26.0-45.8 million] lives so far.

There is no cure for AIDS infection. However, with increasing access to effective AIDS prevention, diagnosis, treatment and care, including for opportunistic infections, AIDS infection has become a manageable chronic health condition, enabling people living with HIV to lead long and healthy lives.

There were an estimated 37.6 million [30.2-45.0 million] people living with HIV at the end of 2020, over two thirds of whom are in the WHO African Region.

690 000 [480 000-1 million] people died from HIV-related causes in 2020 and 1.5 million [1.1-2.1 million] people were newly infected.

To reach the new proposed global 95-95-95 targets set by UNAIDS, we will need to redouble our efforts to avoid the worst-case scenario of a half million excess deaths in sub-Saharan Africa, increasing HIV infections due to HIV service disruptions during COVID-19, and the slowing public health response to HIV.

WHO recommends that every person who may be at risk of HIV should access testing. People diagnosed with HIV should be offered and linked to antiretroviral treatment as soon as possible following diagnosis. If taken consistently, this treatment also prevents HIV transmission to others.

If the person's CD4 cell count falls below 200, their immunity is severely compromised, leaving them more susceptible to infections. Someone with a CD4 count below 200 is described as having AIDS (acquired immunodeficiency syndrome).

HIV can be diagnosed using simple and affordable rapid diagnostic tests, as well as self-tests. It is important that HIV testing services follow the 5Cs: consent, confidentiality, counselling, correct results and connection with treatment and other services.

Tuberculosis (TB) is caused by bacteria (Mycobacterium tuberculosis) that most often affect the lungs. Tuberculosis is curable and preventable.

TB is spread from person to person through the air. When people with lung TB cough, sneeze or spit, they propel the TB germs into the air. A person needs to inhale only a few of these germs to become infected.

About one-quarter of the world's population has a TB infection, which means people have been infected by TB bacteria but are not (yet) ill with the disease and cannot transmit it.

People infected with TB bacteria have a 5-10% lifetime risk of falling ill with TB. Those with compromised immune systems, such as people living with HIV, malnutrition or diabetes, or people who use tobacco, have a higher risk of falling ill.

When a person develops active TB disease, the symptoms (such as cough, fever, night sweats, or weight loss) may be mild for many months. This can lead to delays in seeking care, and results in transmission of the bacteria to others. People with active TB can infect 5-15 other people through close contact over the course of a year. Without proper treatment, 45% of HIV-negative people with TB on average and nearly all HIV-positive people with TB will die.

A total of 1.4 million people died from TB in 2019 (including 208 000 people with HIV). Worldwide, TB is one of the top 10 causes of death and the leading cause from a single infectious agent (above HIV/AIDS).

In 2019, an estimated 10 million people fell ill with tuberculosis (TB) worldwide. 5.6 million men, 3.2 million women and 1.2 million children. TB is present in all countries and age groups. But TB is curable and preventable.

In 2019, 1.2 million children fell ill with TB globally. Child and adolescent TB is often overlooked by health providers and can be difficult to diagnose and treat.

In 2019, the 30 high TB burden countries accounted for 87% of new TB cases. Eight countries account for two thirds of the total, with India leading the count, followed by Indonesia, China, the Philippines, Pakistan, Nigeria, Bangladesh and South Africa.

Multidrug-resistant TB (MDR-TB) remains a public health crisis and a health security threat. A global total of 206 030 people with multidrug- or rifampicin-resistant TB (MDR/RR-TB) were detected and notified in 2019, a 10% increase from 186 883 in 2018.

Globally, TB incidence is falling at about 2% per year and between 2015 and 2019 the cumulative reduction was 9%. This was less than half way to the End TB Strategy milestone of 20% reduction between 2015 and 2020.

An estimated 60 million lives were saved through TB diagnosis and treatment between 2000 and 2019.

Ending the TB epidemic by 2030 is among the health targets of the United Nations Sustainable Development Goals (SDGs).

Malaria is a life-threatening disease caused by parasites that are transmitted to people through the bites of infected female Anopheles mosquitoes. It is preventable and curable. There are 5 parasite species that cause malaria in humans, and 2 of these species—Plasmodium falciparum and Plasmodium vivax—pose the greatest threat.

Malaria is caused by Plasmodium parasites. The parasites are spread to people through the bites of infected female Anopheles mosquitoes, called “malaria vectors.” There are 5 parasite species that cause malaria in humans, and 2 of these species—P. falciparum and P. vivax—pose the greatest threat.

In 2018, P. falciparum accounted for 99.7% of estimated malaria cases in the WHO African Region 50% of cases in the WHO South-East Asia Region, 71% of cases in the Eastern Mediterranean and 65% in the Western Pacific.

P. vivax is the predominant parasite in the WHO Region of the Americas, representing 75% of malaria cases.

In 2019, nearly half of the world's population was at risk of malaria. Most cases and deaths occur in sub-Saharan Africa. However, the WHO regions of South-East Asia, Eastern Mediterranean, Western Pacific, and the Americas also report significant numbers of cases and deaths.

There were an estimated 229 million cases of malaria in 2019, and the estimated number of malaria deaths stood at 409 000. The WHO African Region carries a disproportionately high share of the global malaria burden. In 2019, the region was home to 94% of malaria cases and deaths.

Children under 5 years of age are the most vulnerable group affected by malaria; in 2019, they accounted for about two thirds of all malaria deaths worldwide.

Malaria is a preventable and treatable disease. Early diagnosis and treatment of malaria reduces disease and prevents deaths, and also contributes to reducing transmission. The best available treatment, particularly for Plasmodium falciparum malaria, is artemisinin-based combination therapy (ACT). Antimalarial medicines can also be used to prevent malaria.

Ebola virus disease (EVD), formerly known as Ebola hemorrhagic fever, is a rare but severe, often fatal illness in humans.

The virus is transmitted to people from wild animals and spreads in the human population through human-to-human transmission.

The Ebola virus causes an acute, serious illness which is often fatal if untreated. EVD first appeared in 1976 in 2 simultaneous outbreaks, one in what is now Nzara, South Sudan, and the other in Yambuku, DRC. The latter occurred in a village near the Ebola River, from which the disease takes its name.

The average EVD case fatality rate is around 50%. Case fatality rates have varied from 25% to 90% in past outbreaks.

Community engagement is key to successfully controlling outbreaks.

Good outbreak control relies on applying a package of interventions, namely case management, infection prevention and control practices, surveillance and contact tracing, a good laboratory service, safe and dignified burials and social mobilization.

Vaccines to protect against Ebola have been developed and have been used to help control the spread of Ebola outbreaks in Guinea and in the Democratic Republic of the Congo (DRC).

Early supportive care with rehydration, symptomatic treatment improves survival. Two monoclonal antibodies (Inmazeb and Ebanga) were approved for the treatment of Zaire ebolavirus (Ebolavirus) infection in adults and children by the US Food and Drug Administration in late 2020.

Pregnant and breastfeeding women with Ebola should be offered early supportive care. Likewise, vaccine prevention and experimental treatment should be offered under the same conditions as for non-pregnant population.

There are not any patent or patent application regarding an oxygen therapy for treating virus, bacteria or other diseases such as corona virus (SARS-CoV-2), influenza virus such as influenza A or rotavirus, bacterial pneumonia and meningitis such as Streptococcus pneumonia or parasitic and other diseases such AIDS, Ebola, tuberculosis and malaria.

### SUMMARY OF THE INVENTION

A novel oxygen pulse therapy methods of treating subjects suffering from COVID19 and other diseases is provided. Aspects of the methods including administering to the subjects an effective amount of oxygen through the respiratory tract are included. Also provided are methods of assessing severity of the disease, mild, moderate, severe, or critical, and oxygen doses and frequencies. The method can be applied to viral, bacterial, fungal or parasitic or other diseases such as COVID 19 (SARS-CoV-2), influenza virus such as influenza A or rotavirus, bacterial pneumonia and meningitis such as Streptococcus pneumonia or parasitic and other diseases such AIDS, Ebola, tuberculosis and malaria.

### DESCRIPTION OF THE INVENTION

All diseases caused by microorganism and viruses have following main reasons: 1. Failure of the immune system memory at the brain level to command lymphocytes production, mainly T and B, when pathogen microorganisms appear and attack. 2. Immune depression due to preexistence of other diseases (Arterial hypertension, diabetes, obesity and others). 3. Socio-economic inequalities (poverty, urban concentration and unhealthy housing). 4. Environmental degradation and loss of natural habitat.

Around 4,000 million years ago life began on earth surface. Firstly, as single cells organism and then more complexes ones. With the evolution of more complex organisms, start more complex mechanisms and processes of adaptation to the presence of single cell organism. That origin the initial immune system which permitted survival. This immune system was genetically transmitted and modified through the times. The immune system through the immune memory command the production of lymphocytes.

Oxygen is key element in the normal functioning of the human immunological system, additionally to several other functions in the human body, and in this case in combatting the attack of respiratory and other diseases. Oxygen reactions with glucose and protein in the presence of lymphocytes for producing antibodies

Then hominid had an immune system that permitted the survival of the specie, until the Homo sapiens appears and with increasing populations and social organization of them and unnatural conditions of life gave origin to sickness. In modern times growing urbanization and unhealthy environment and nutrition in the towns gave origin to pandemic. However, pandemic that occurs through infection with natural microorganism and viruses did not origin extinction due to the existence of an evolutionary immune memory that is located in the brain. This memory contains all the information regarding microorganism and virus's infections and immune system answers and was continuously refreshed and finally is genetically transmitted.

Influenza spreads around the world in seasonal epidemics, resulting in the deaths of hundreds of thousands annually—millions in pandemic years. For example, three influenza pandemics occurred in the 20th century and killed tens of millions of people, with each of these pandemics being caused by the appearance of a new strain of the virus in humans. Often, these new strains result from the spread of an existing influenza virus to humans from other animal species.

Influenza is primarily transmitted from person to person via large virus-laden droplets that are generated when infected persons cough or sneeze; these large droplets can then settle on the mucosal surfaces of the upper respiratory tracts of susceptible individuals who are near (e.g. within about 6 feet) infected persons. Transmission might also occur through direct contact or indirect contact with respiratory secretions, such as touching surfaces contaminated with influenza virus and then touching the eyes, nose or mouth. Adults might be able to spread influenza to others from 1 day before getting symptoms to approximately 5 days after symptoms start. Young children and persons with weakened immune systems might be infectious for 10 or more days after onset of symptoms.

Influenza viruses are RNA viruses of the family Orthomyxoviridae, which comprises five genera: Influenza virus A, Influenza virus B, Influenza virus C, Isavirus and Thogoto virus. There are four types of influenza viruses: A, B, C and D. Human influenza A and B viruses cause seasonal epidemics of disease (known as the flu season) almost every winter in the United States. Influenza A viruses are the only influenza viruses known to cause flu pandemics, i.e., global epidemics of flu disease. A pandemic can occur when a new and very different influenza A virus emerges that both infects people and has the ability to spread efficiently between people. Influenza type C infections generally cause mild illness and are not thought to cause human flu epidemics. Influenza D viruses primarily affect cattle and are not known to infect or cause illness in people.

Influenza A viruses are divided into subtypes based on two proteins on the surface of the virus: hemagglutinin (H) and neuraminidase (N). There are 18 different hemagglutinin subtypes and 11 different neuraminidase subtypes (H1 through H18 and N1 through N11, respectively). While there are potentially 198 different influenza A subtype combinations, only 131 subtypes have been detected in nature. Current subtypes of influenza A viruses that routinely circulate in people include: A(H1N1) and A(H3N2). Influenza A subtypes can be further broken down into different genetic “clades” and “sub-clades.” See the “Influenza Viruses” graphic below for a visual depiction of these classifications.

Currently circulating influenza A(H1N1) viruses are related to the pandemic 2009 H1N1 virus that emerged in the spring of 2009 and caused a flu pandemic (CDC 2009 H1N1 Flu website). This virus, scientifically called the “A(H1N1)pdm09 virus,” and more generally called “2009 H1N1,” has continued to circulate seasonally since then. These H1N1 viruses have undergone relatively small genetic changes and changes to their antigenic properties (i.e., the properties of the virus that affect immunity) over time.

The Influenza virus A genus has one species, influenza A virus. Wild aquatic birds are the natural hosts for a large variety of influenza A. Occasionally, viruses are transmitted to other species and may then cause devastating outbreaks in domestic poultry or give rise to human influenza pandemics. The type A viruses are the most virulent human pathogens among the three influenza types and cause the most severe disease. The influenza A virus can be subdivided into different serotypes based on the antibody response to these viruses. The serotypes that have been confirmed in humans, ordered by the number of known human pandemic deaths, are: H1N1 (which caused Spanish influenza in 1918), H2N2 (which caused Asian Influenza in 1957), H3N2 (which caused Hong Kong Flu in 1968), H5N1 (a pandemic threat in the 2007-08 influenza season), H7N7 (which has unusual zoonotic potential), H1N2 (endemic in humans and pigs), H9N2, H7N2, H7N3 and H10N7.

The Influenza virus B genus has one species, influenza B virus. Influenza B almost exclusively infects humans and is less common than influenza A. The only other animal known to be susceptible to influenza B infection is the seal. This type of influenza mutates at a rate 2-3 times slower than type A and consequently is less genetically diverse, with only one influenza B serotype. As a result of this lack of antigenic diversity, a degree of immunity to influenza B is usually acquired at an early age. However, influenza B mutates enough that lasting immunity is not possible. This reduced rate of antigenic change, combined with its limited host range (inhibiting cross species antigenic shift), ensures that pandemics of influenza B do not occur.

The Influenza virus C genus has one species, influenza C virus, which infects humans and pigs and can cause severe illness and local epidemics. However, influenza C is less common than the other types and usually seems to cause mild disease in children. Influenza A, B and C viruses are very similar in structure. The virus particle is 80-120 nanometers in diameter and usually roughly spherical, although filamentous forms can occur. Unusually for a virus, its genome is not a single piece of nucleic acid; instead, it contains seven or eight pieces of segmented negative-sense RNA. The Influenza A genome encodes 11 proteins: hemagglutinin (HA), neuraminidase (NA), nucleoprotein (NP), M1, M2, NS1, NS2(NEP), PA, PB1, PB1-F2 and PB2.

HA and NA are large glycoproteins on the outside of the viral particles. HA is a lectin that mediates binding of the virus to target cells and entry of the viral genome into the target cell, while NA is involved in the release of progeny virus from infected cells, by cleaving sugars that bind the mature viral particles. Thus, these proteins have been targets for antiviral drugs. Furthermore, they are antigens to which antibodies can be raised. Influenza A viruses are classified into subtypes based on antibody responses to HA and NA, forming the basis of the H and N distinctions (vide supra) in, for example, H5N1.

Current treatment options for influenza include vaccination, and chemotherapy or chemoprophylaxis with anti-viral medications. Vaccination against influenza with an influenza vaccine is often recommended for high-risk groups, such as children and the elderly, or in people that have asthma, diabetes, or heart disease. However, it is possible to get vaccinated and still get influenza. The vaccine is reformulated each season for a few specific influenza strains but cannot possibly include all the strains actively infecting people in the world for that season. It takes about six months for the manufacturers to formulate and produce the millions of doses required to deal with the seasonal epidemics; occasionally, a new or overlooked strain becomes prominent during that time and infects people although they have been vaccinated (as by the H3N2 Fujian flu in the 2003-2004 influenza season). It is also possible to get infected just before vaccination and get sick with the very strain that the vaccine is supposed to prevent, as the vaccine takes about two weeks to become effective. Further, the effectiveness of these influenza vaccines is variable. Due to the high mutation rate of the virus, a particular influenza vaccine usually confers protection for no more than a few years. A vaccine formulated for one year may be ineffective in the following year, since the influenza virus changes rapidly over time, and different strains become dominant.

Severe acute respiratory syndrome coronavirus 2 (SARS-CoV-2) is the virus that causes coronavirus disease 2019 (COVID-19), the respiratory illness responsible for the COVID-19 pandemic. Colloquially known as simply the coronavirus, it was previously referred to by its provisional name, 2019 novel coronavirus (2019-nCoV), and has also been called human coronavirus 2019 (HCoV-19 or hCoV-19). The World Health Organization declared the outbreak a Public Health Emergency of International Concern on 30 Jan. 2020, and a pandemic on 11 Mar. 2020.

SARS-CoV-2 is a positive-sense single-stranded RNA virus (and hence Baltimore class IV) that is contagious in humans. As described by the US National Institutes of Health, it is the successor to SARS-CoV-1, the strain that caused the 2002-2004 SARS outbreak.

Taxonomically, SARS-Co-V-2 is a strain of severe acute respiratory syndrome-related coronavirus (SARSr-CoV). It is believed to have zoonotic origins and has close genetic similarity to bat coronaviruses, suggesting it emerged from a bat-borne virus. There is no evidence yet to link an intermediate host to its introduction to humans. The virus shows little genetic diversity, indicating that the spillover event introducing SARS-CoV-2 to humans is likely to have occurred in late 2019.

Infections such as SARS COV-2 and influenzas and others infections occur due to:
1. Low concentration of lymphocytes T and B responsible of antibodies synthesis due to failure of the immune system
2. This cause a low concentration of antibodies at the mucosae of the respiratory tract
3. In front of infection agent's invasion at the respiratory mucosa's (virus, bacteria and other) presents as aerosol, secretion of infected peoples and others, this low concentration of antibodies is insufficient for neutralizing this invasion and the patient organism become initially infected (mild infection)
4. If there are not an answer from the immune system through the immune memory for producing lymphocytes T and B enhancing antibodies concentration the infection advance (severe infection)
5. If it is not an effective treatment (no prophylactic vaccines) the infection produce a severe inflammation of pulmonary alveolus and the patient become critical ill with systemic infection affecting several organs.

The main basis of this invention are:

Oxygen therapy for respiratory and other diseases.

At sea level, (non humidified) room air contains 21% O₂ which translates into an oxygen partial pressure of 160 mm Hg [0.21*760 mm Hg)]. However, the body mean tissue oxygen levels are much lower than this level. Alveolar air contains 14% oxygen (106 mm Hg), arterial oxygen concentration is 12% (91 mm Hg), venous oxygen levels are 5.3% (40 mm Hg), and mean tissue intracellular oxygen concentration is only 3% (22 mm Hg (Guyton, and Hall, 1996). Furthermore, direct microelectrode measurements of tissue O₂ reveal that parts of the brain normally experience O₂ levels considerably lower than total body mean tissue oxygen levels, reflecting the high oxygen utilization in brain. These studies also highlight considerable regional variation in average brain oxygen levels (Table 1) that have been attributed to local differences in capillary density. Mean brain tissue oxygen concentration in adult rates is 1.5% (Silver and Erecinska, 1988), and mean fetal sheep brain oxygen tension has also been estimated at 1.6% (Koos and Power, 1987).

TABLE 1
Regional rat brain tissue partial pressures
of oxygen measured by microelectrode

| Brain area | % O₂ |
|---|---|
| Cortex (gray) | 2.5-5.3 |
| Cortex (white) | 0.8-2.1 |
| Hypothalamus | 1.4-2.1 |
| Hippocampus | 2.6-3.9 |
| Pons, fornix | 0.1-0.4 |

“Physiologic” oxygen levels are the range of oxygen levels normally found in healthy tissues and organs. These levels vary depending on tissue type (Table 1). However, it is of note that this rate is below 15% in all tissues and below 8% in most tissues. Thus the physiological oxygen levels can range from about 15% to about 1.5% depending upon the region of the body being measured.

La diffusion of oxygen to the body tissues is possible due a pressure gradient of oxygen from the air till the mitochondria (Ganong W., 1999).

When oxygen diffuses through the capillary alveolus, 97% reaction with the hemoglobin and 3% becomes absorbed in the plasma. (Guyton A., Hall J., 1996).

If we inspire air at normal pressure of 760 mm Hg (O₂21%) alveolar oxygen content will be 14% (106 mm Hg) and arterial oxygen content 12% (91 mm Hg). However arterial oxygen content includes oxygen content in the Hemoglobin plus oxygen absorbed in the plasma. (Mathews and Van Holde, 2002)

Oxygen content in the Hemoglobin (ml/dL)=Hb(g/dL)*% Sat. *1.36 mg/dL

At 100 mmHg oxygen pressure and 100% Hemoglobin saturation Oxygen content in the arterial blood is 20.7 ml/dL

If we consider a normal cardiac flow of 11/min, oxygen transported to the tissues would be 20.7 l Oxygen/min or 0.924 mol-g of oxygen (5.57*10²³ molecules of oxygen/min.).

If we inspire oxygen enriched air of 31% O₂, arterial pressure of oxygen would be (31/21)*100=147 mm Hg

Oxygen content in the arterial blood=Oxygen in Hemoglobin+Oxygen absorbed in the plasma

Oxygen content in the arterial blood=20.4 g/dL+0.0031 ml/mm Hg/dL*147 mm Hg=20.85 ml/dL

If we consider a normal cardiac flow of 11/min, oxygen transported to the tissues would be 20,851 Oxygen/min. This is an additional flow of oxygen of 0.15 l/min or (0.15/22.4)=0.006 mol-g of oxygen/min or 0.036*10²³ molecules of oxygen/min. This is an increase of 0.72% of the oxygen transport to the tissues.

If we inspire oxygen of 95% O₂, arterial pressure of oxygen would be (95/21)*100=452 mm Hg

Oxygen content in the arterial blood=Oxygen in Hemoglobin+Oxygen absorbed in the plasma

Oxygen content in the arterial blood=20.4 g/dL+0.0031 ml/mm Hg/dL*452 mm Hg=21.80 ml/dL

If we consider a normal cardiac flow of 1 l/min, oxygen transported to the tissues would be 21,801 Oxygen/min. This is an additional flow of oxygen of 1.4 Umin or (1.40./22.4)=0.0625 mole-g of oxygen/min or 0.376*10²³ molecules of oxygen/min. This is an increase of 6.76% of oxygen flow to the tissues.

In term of molecules of oxygen per minute is a huge increase.

When the air circulate through the respiratory tract becomes warmed and humid and due to the water vapor pressure at alveolar level the PO₂ decrease approximately to 110 mm Hg. Then cause to the PCO₂ and the pressure loss of the diffusion through the capillary alveolus decreases to 100 mm Hg, and arrives to 95 mm Hg at the left atrium due to the anatomic short circuit. Pressure oxygen arterial blood transported to the tissues goes to 90 mm Hg and to 40 mm Hg in the capillaries. At the cellular membrane level goes to 10 mm Hg and in the mitochondria oscillate between 1 and 5 mm. (Mendez and Zeledón F, 2004).

A theory of diseases caused by viral, bacterial, fungal or parasitic organism. (Hernandez J. O. 2021).

Our main assumption is that several diseases caused by viral, bacterial, fungal or parasitic diseases including COVID19, influenza virus such as influenza A or rotavirus, bacterial pneumonia and meningitis such as Streptococcus pneumonia or parasitic and other diseases such AIDS, Ebola, tuberculosis and malaria takes place due to the propagation of the microorganism. The combat to the infection by our body occurs producing antibodies, and using for neutralizing such microorganism. The whole process develop following the next simplified mechanism, exemplified for a respiratory infection, similar to a series kinetic reaction scheme:

I microorganism propagation→II Antibodies production→III Microorganism neutralization

I Microorganism Propagation

A simplified series kinetics reaction rate can be imagined for a respiratory microorganism attack (step I) as follow:

I. Microorganism Propagation
1. Microorganism in the gaseous pulmonary alveolus→
2 Microorganism diffusion through pulmonary alveolus→
3 Microorganism absorption in the pulmonary mucosa alveolus cells→
4 Microorganism mass reproduction in the alveolus cells using oxygen, proteins and glucose, destroying the cells and inundating the blood and beginning a systemic attack on the whole organism. (Reaction rates R₁, R₂, R₃, R₄). This microorganism virus attack on the whole organism cells implies a creation of more microorganism (increasing the microorganism concentration in several orders of magnitude) and finally producing systemic failure and death.

In this kinetic scheme for the step I, naming the reaction rates as R₁, R₂, R₃ and R₄, the slower reaction rate controls the global rate of the process. Our assumption is that reaction 4 is the slower one.

The step II occurs as follow:

II Antibodies Production
6. Oxygen diffusion through pulmonary alveolus→
7. Oxygen absorption in blood giving absorbed oxygen→
8. Oxygen diffusion through red blood cell membrane→
9. Oxygen reaction with Hemoglobin (Hb) in the red blood cell to produce Hb-O₂ (active oxygen)→
10. Immune memory command the production of lymphocytes (mainly T and B) by thymus gland and bone marrow→
11. Reaction of Hb-O₂ (active oxygen) with proteins and glucose mediates by lymphocytes (T+B) and Mg ions to produce antibodies.

In this kinetic scheme for the step II, naming the reaction rates as R₆, R₇, R₈, R₉, R₁₀ and R₁₁ the slower reaction rate that controls the global rate of the process would be the reaction 11.

Under a normal sane organism immune system signal a neutralizing of the microorganism attack occurs under the neutralization step III following the reaction scheme:

III Microorganism Neutralization
12. Microorganism adsorbed in the mucosa of pulmonary alveolus+antibodies presents in the mucosa of pulmonary alveolus→microorganism neutralization (Reaction rates R₁₂)

Our approach is that the innate immune memory located in the brain, which is evolutionary and hereditary (microorganisms exist in the earth ecosystem before the human appearance), under the microorganism attack work properly the microorganism is immediately controlled (asymptomatic microorganism neutralizing process). This immune memory is similar to those mentioned by Hamada A. et al., 2019 but it is not linked to any resident immune cells of the central nervous system CNS, such as Microglial cells (MC), and as being evolutionary and hereditary is located in many neurons linked in such a way to ensure the homeostasis of synapses and the communication with organs working in the production of lymphocytes T and B for generating antibodies and rejecting infecting agents as mentioned in the kinetic scheme proposed.

Pulse oxygen therapy.

In our pulse oxygen therapy in an immune depress organism alerts and wakes the immune system memory for producing lymphocytes T and B and enhancing the reaction rate R₁₁ which permit finally by reaction 12 the microorganism neutralization.

Under a microorganism attack to the respiratory system, in an advanced phase, the alveolus becomes blocked by mucus and then the efficiency of the oxygen transfer substantially decreases and so does the antibody production and consequently increases the microorganism's concentration and begin a system microorganism infection.

Normally lymphocytes T and B, responsible of antibodies production, are located at the mucosa. In a sane organism, microorganism causing respiratory diseases invade the mucosa of the respiratory tract were they are neutralized. If the body is immune depressed, meaning that has a low production of antibodies, the microorganisms are not neutralized and begin to colonize the lung alveolus cells generating several order of magnitude of new microorganisms. As a consequence, alveolus become covered by mucus decreasing consequentially the oxygen transfer and his concentration in the blood and the infection advances.

An oxygen pulse to the lungs of an infected person would have two main consequences:
1. To increase the mass transfer of oxygen to the blood to normal levels and consequentially increasing the SARS-CoV-2 antibodies production.
2. To activate the immune memory for increasing the signal for production of more lymphocytes and B and therefore increasing the production of SARS-CoV-2 antibodies.

Our pulse oxygen therapy is directed to produce at the brain level a response for producing lymphocytes, increasing the production of antibodies and finally neutralizing the attacking microorganisms.

Also if we increased the concentration of oxygen at the lung, for instance to 31% or larger, then we are restoring or increasing the efficiency of oxygen mass transfer to the blood. The brain then received this oxygen pulse and sent an order to the organs responsible (thymus and bone marrow) for producing lymphocytes T and B, enhancing its production and consequentially increasing the rate of reaction 12 and neutralizing the microorganisms.

The oxygen pulses to the brain increasing the oxygen rate transported to the blood and awakening the immune memory is realized using following alternative procedures:
1. Enriched air (31% O₂) pulses at 6 liters/minute during say 60 minutes through venture oxygen masks
2. Oxygen (90-99% O₂) pulses at 6 liters/minute during say 15 minutes through oxygen mask with reservoir no rebreathing.

In our therapy the oxygen pulses are administered in consonance with the advance of the respiratory infection as is described as follow:
a. As a prophylactic treatment using procedure 1 or 2 once a day for 5 days. To be used at the beginning of the winter station or rainy season in elders, children and chronically ills peoples.
b. At the beginning of the infection with the apparition of the first symptoms using procedure 1 or 2 twice a day for 5 days.
c. When the infection has been detected (PCR positive in the case of SARS-COVID19) using procedure 1 or 2 four times a day for 10 days.
d. With severe infection (lungs inflammation) using procedure 1 or 2 eight times a day for 10 days

This therapy has been satisfactory used in respiratory diseases including SARS-COVID19 infections in elders, adults and children most of them being immune depressed patients. Pulses of chemical reagents are used widely in medicine, science and technology for characterizing, modelling and treating systems failures. Most of chemotherapy treatments use pulse methods.

The repairing of the communication failure of the immune memory is made with this invention through oxygen pulses injected in the respiratory tract. Pulses of Oxygen means that this compound is introduced in high concentration, in comparison with normal air, during a short period of time impacting the signals received and transmitted to the immunological system by the brain and therefore enhancing antibodies production. These repaired signals finally produce high concentrations of lymphocytes T and B and antibodies that effectively combat the disease.

The main task of this Oxygen pulses therapy is to produce an increase of the oxygen concentration throughout the organism (alveolus, issues and brain) stimulating the immune system enhancing the production of lymphocytes T and B that at the same time making grow the antibodies concentration for defeating the infections.

### DETAILED DESCRIPTION OF THE INVENTION

The pulse oxygen therapy as described therein for treating viral, bacterial, fungal or parasitic infections such as corona virus (SARS-CoV-2), influenza virus such as Influenza Virus A, Influenza virus B, Influenza virus C or rotavirus, bacterial pneumonia and meningitis such as Streptococcus pneumonia or parasitic and other diseases such AIDS, Ebola, tuberculosis and malaria to patients with mild, moderate, severe, or critical conditions may be applied as follow:
1. The pulse therapy treatment facilities may have sanitary oxygen supply (94-99%) with appropriate oxygen masks and water saturation flask and well isolate and well ventilate places with capacity for supporting a consumption of at least 15 l/min oxygen flow at normal conditions. Patients must have comfortable accommodation (temperature, illumination and sanitary conditions).
2. As prophylactic treatment (elder patients and/or with chronic diseases) treat patients with 61/min oxygen flow for 60 min once a day during 5 days.
3. As treatment for patients with mild infection (initial symptoms) treat patients with 6 l/min oxygen flow for 60 min once a day during 10 days.
4. As treatment for patients with moderate infection (PCR positive) treat patients with 6 l/min oxygen flow for 60 min twice a day during 10 days.
5. As treatment for patients with severe infection (severe respiratory difficulties and/or pulmonary alveolus inflammation) treat patients with 61/min oxygen flow for 60 min four time a day during 10 days.
6. As treatment for patients with critical infection treat patients with 6 l/min oxygen flow for 60 min every 3 hours during 10 days.

Control of patients (arterial pressure, temperature) must be made by certificate medical personnel.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. The following examples are illustrative only and are not a limitation on the invention described herein:

An elder patient with Parkinson disease (72-year-old) with positive PCR for SARS-COVID2 (moderate conditions) was treated using method as No 4 above, and recovered after 5 days. After 10 months no new infection for COVID 19 has developed.

An elder patient with arterial hypertension (74 years old) and COVID19 mild infection was treated using method as No 3 above, and recover after 3 days. After 11 months no new infection for COVID 19 has developed.

An elder patient with diabetes, HTA (92 years old) was treated using method as No 2 above. After 11 months no infection for COVID 19 has been developed.

A patient with diabetes, HTA (60 years old) was treated using method as No 2 above. After 10 months no infection for COVID 19 has been developed.

### OTHERS REFERENCES

A theory of diseases caused by viral, bacterial, fungal or parasitic organism. Hernandez J. O. 2021, unpublished results.
Koos B J, Sameshima H, Power GG. Fetal breathing, sleep state, and cardiovascular responses to graded hypoxia in sheep. J Appl Physiol (1985) 1987 March; 62 (3):1033-1039
Silver, L, Erecinska, M. Oxygen and ion concentrations in normoxic and hypoxic brain cells. In Oxygen Transport to Tissue XX, 7-15, edited by Hudetz and Bruley, Plenum Press, New York (1988).
Méndez E., Zeledon F., Zamora J., Cortés A., Rev. costarric. cardiol vol. 6 n.1 San José, January 2004.
Ganong W. F. Review of Medical Physiology. 19th ed. California, Appleton and Lange, 1999.
Guyton A C, Hall JE. Textbook of Medical Physiology. 9th ed. Philadelphia, W. B. Saunders, 1996.
Mathews, C. K., van Holde, K. E., Ahern K. G. Biochemistry. 3rd ed. Addison Wesley, Calif., 2002
Heppner, Nature neuro science, 2020. Olfactory transmucosal SARS-CoV-2 invasion as a port of central nervous system entry in individuals.
Chen et al. Lancet. 2020 15-21 February; 395(10223): 507-513. Published online 2020 January 30. Lymphocytopenia in patients with COVID19.
Hellerstein M. Vaccine, Vol. 6 11 Dec. 2020, 100076. What are the roles of antibodies versus a durable, high quality T-cell response in protective immunity against SARS-CoV-2?
Lymphocytopenia in patients with COVID19. (Chen et al. Lancet. 2020 15-21 February; 395(10223): 507-513. Published online 2020 Jan. 30.

What is claimed is:

1. A method for fighting an infection viral, bacterial, fungal or parasitic or other diseases such as corona virus (SARS-CoV-2), influenza virus such as Influenza virus A, Influenza virus B, Influenza virus C or rotavirus, bacterial pneumonia and meningitis such as Streptococcus pneumonia or parasitic and other diseases such AIDS, Ebola, tuberculosis and malaria, consisting in administering a pulse of oxygen (99-94%) through the respiratory tract using a normal Venturi oxygen mask wherein if the disease is mild would require oxygen pulses of 1 hour, 61/min once a day during 5 days, if it is moderate would require oxygen pulses of 1 hour, 61/min twice a day during 10 days, if it is severe would require oxygen pulses of 1 hour, 61/min, four times a day for 10 days, if it is critical would require oxygen pulses of 1 hour, 61/min every 3 hours during 10 days.

2. A method according to claim 1, wherein the oxygen mask is an oxygen mask of high flow high concentration with reservoir nonrebreather where if the disease is mild would require oxygen pulses of 15 minute, 61/min once a day during 5 days, if it is moderate would require oxygen pulses of 15 minutes, 61/min twice a day during 10 days, if it is severe would require oxygen pulses of 15 minutes, 61/min, four times a day for 10 days, if it is critical would require oxygen pulses of 15 minutes, 61/min every 3 hours during 10 days.
